(19) United States
(12) Patent Application Publication
(10) Pub. No.: US 2004/0092767 A1
(43) Pub. Date: May 13, 2004

(54) Process for the production of an aqueous solution of adipic acid, hydroxyhexanoic acid and oligomers thereof

(76) Inventors: Luis A. Chu, Forsyth, IL (US); Ludovic Fodor, Beaumont, TX (US); David L. Valdez, Victoria, TX (US)

Correspondence Address: E I DU PONT DE NEMOURS AND COMPANY, LEGAL PATENT RECORDS CENTER, BARLEY MILL PLAZA 25/1128, 4417 LANCASTER PIKE, WILMINGTON, DE, 19805, US

(21) Appl. No.: 10/289,572

(22) Filed: Nov. 7, 2002

(51) Int. Cl. C07C 51/31

(52) U.S. Cl. 562/543

(57) ABSTRACT

Process for producing an aqueous solution comprising adipic acid, hydroxyhexanoic acid and oligomers thereof.

### BACKGROUND OF THE INVENTION

Liquid phase reaction of cyclohexane with oxygen produces an oxidation product comprising cyclohexanol (A), cyclohexanone (K) and cyclohexyl hydroperoxide (CHHP) (British 777,087, U.S. Pat. No. 3,530,185 & U.S. Pat. No. 3,957,876). Other products of the above oxidation process include adipic acid (AA), 6-hydroxyhexanoic acid (HYCAP), 6-hydroperoxyhexanoic acid (HCHP) and water (U.S. Pat. No. 3,719,706, U.S. Pat. No. 3,726,917 & U.S. Pat. No. 6,288,286).

The oxidation product can be contacted with water to increase the amount of water in the oxidation process to between 1 and 5 wt %. The water supplemented oxidation product can then be separated into an aqueous phase and an organic phase (U.S. Pat. No. 3,726,917). The aqueous phase comprises the majority of the AA, HYCAP, HCHP and a portion of CHHP.

The aqueous phase can be added back into the downstream of the process. At the end of the process the AA, HYCAP, and water content of the aqueous phase is separated from the final product of A and K to form a hazardous waste stream that is required to be disposed of in a safe and environmentally friendly method. This involves additional costs and environmental concerns.

It would be desirable to have a process of cyclohexane oxidation that will eliminate the requirement to form this separate hazardous waste stream and the costs associated with disposing of the hazardous waste. It would also be desirable if at least a portion of the stream forming the hazardous waste can be converted to a saleable product.

### SUMMARY OF THE INVENTION

In the present invention, the aqueous stream comprising AA and HYCAP is further treated to produce a saleable aqueous product. Therefore, the cost of disposal of the aqueous stream (hazardous waste) is reduced or eliminated.

The present invention is, therefore, a process for the production of an aqueous solution comprising adipic acid, hydroxyhexanoic acid and oligomers thereof, said process comprising:

a. contacting cyclohexane with an oxygen-containing gas to produce an oxidation product that comprises cyclohexane, cyclohexyl hydoperoxide, cyclohexanone, cyclohexanol, hydroperoxyhexanoic acid, adipic acid, hydroxyhexanoic acid and water;

b. adding water to the oxidation product to produce a water-supplemented oxidation product

c. separating the water-supplemented oxidation product into an organic phase and an aqueous phase that comprises adipic acid and hydoxyhexanoic acid;

d. heating the aqueous phase at a temperature between 120 and 130 degrees C. and a pressure between 10 and 30 psig (170 kPa and 308 kPa) to evaporate at least a portion of the water in the aqueous phase and decompose at least a portion of the cyclohexyl hydroperoxide and the hydroperoxy hexanoic acid present in the aqueous phase, thereby forming the aqueous solution comprising adipic acid, hydroxyhexanoic acid and oligomers thereof.

### BRIEF DESCRIPTION OF DRAWING

The drawing consists of one figure that depicts a block diagram showing the present invention.

### DETAILED DESCRIPTION OF THE INVENTION

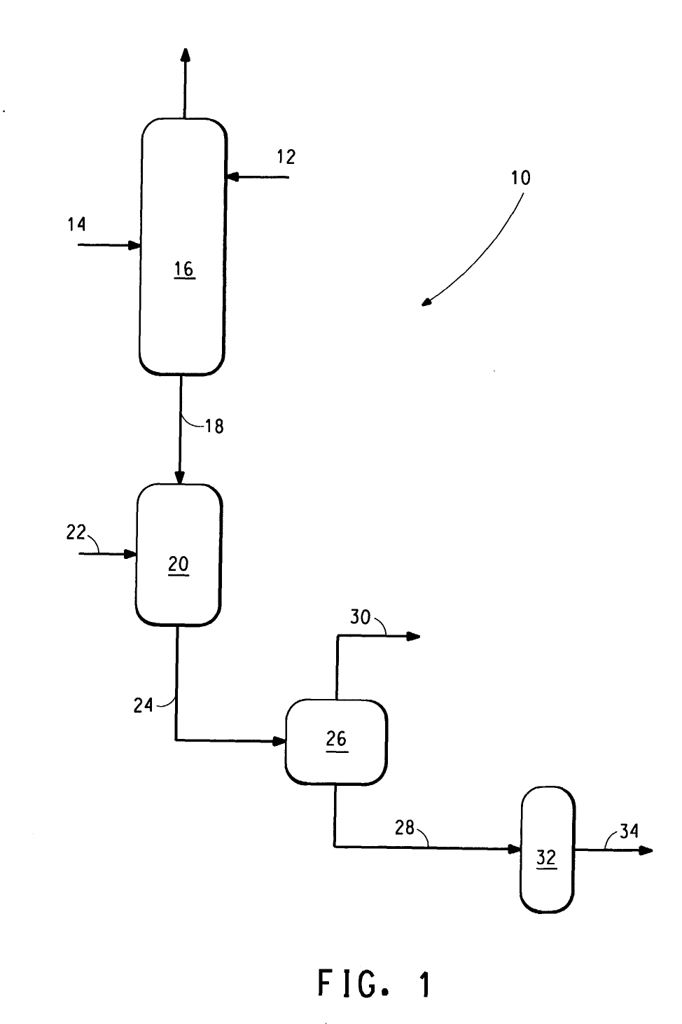

Referring now to FIG. 1, there is shown a block diagram illustrating apparatus 10 that embodies the present invention.

Stream 12 comprising cyclohexane (fresh and recycle) and stream 14 of an oxygen containing gas is fed to an air oxidizer unit 16. The air oxidizer unit is a mass transfer column containing perforated trays containing liquid cyclohexane (U.S. Pat. No. 3,530,185 & U.S. Pat. No. 3,957,876). This allows intimate contact of the oxygen containing gas with liquid cyclohexane. A portion of the cyclohexane is oxidized to CHHP, A and K by this action. In another mode of operation, a series of tanks might be used in place of the tray column.

A stream 18 comprising cyclohexane, CHHP, A and K exits the air oxidizer and is fed to an inline mixer 20). Stream 22 containing water is also fed to the inline mixer 20 in which it is intimately contacted with the organic stream 18 to allow the water to extract the water-soluble components. The mixed aqueous and organic stream 24 exits the inline mixer and is fed to a decanter 26.

The decanter 26 allows separation of the aqueous phase from the organic phase. The organic phase 30 exits the decanter and is processed for recovery of A and K and recycle of cyclohexane (not shown in diagram). The aqueous phase 28 is fed to an evaporator 32. The evaporator concentrates the process stream. A concentration of about 35 to 50 wt. % water is accomplished. Decomposition of at least a portion of the various peroxides is also accomplished in the evaporator. The evaporator can be operated at a pressure between 170 and 308 kPa (10 and 30 psig) and a temperature of 120 to 130° C.

Because of the potential for solids to be present in the aqueous stream 28, dual filters might be provided on the feed line to the evaporator (not shown in diagram) to remove the solids in order to prevent fouling of heat exchangers (not shown in diagram).

The tails stream 34 from the evaporator 32 is pumped through a cooler and filter to process tanks (not shown in diagram). These tanks are designed to provide 2 to 5 days holdup time to decompose the remaining peroxide. The tanks can be operated at a pressure between 101 and 170 kPa (0 and 10 psig) and a temperature between 80 and 100° C. Preferably, the tank temperature is maintained above 80° C. to prevent the precipitation of solids, such as AA.

1. A process for the production of an aqueous solution comprising adipic acid, hydroxyhexanoic acid and oligomers thereof, said process comprising:
a. contacting cyclohexane with an oxygen-containing gas to produce an oxidation product that comprises cyclohexane, cyclohexyl hydoperoxide, cyclohexanone, cyclohexanol, hydroperoxy hexanoic acid, adipic acid, hydroxyhexanoic acid and water;
b. adding water to the oxidation product to produce a water-supplemented oxidation product;
c. separating the water-supplemented oxidation product into an organic phase and an aqueous phase that comprises adipic acid and hydoxyhexanoic acid;
d. heating the aqueous phase at a temperature between 120 and 130 degrees C. and a pressure between 10 and 30 psig (170 kPa and 308 kPa) to evaporate at least a portion of the water in the aqueous phase and decompose at least a portion of the cyclohexyl hydroperoxide and the hydroperoxy hexanoic acid present in the aqueous phase, thereby producing the aqueous solution comprising adipic acid, hydroxyhexanoic acid and oligomers thereof.
